(19) United States
(12) Patent Application Publication
(10) Pub. No.: US 2007/0104676 A1
(43) Pub. Date: May 10, 2007

(54) Cosmetic and/or dermatological preparations containing an extract from the seeds of plants of the genus adenanthera

(75) Inventors: Philippe Moser, Essey-Les-Nancy (FR); Louis Danoux, Saulxures Les Nancy (FR); Gilles Pauly, Nancy (FR)

Correspondence Address: COGNIS CORPORATION;PATENT DEPARTMENT, 300 BROOKSIDE AVENUE, AMBLER, PA, 19002, US

(73) Assignee: COGNIS FRANCE S.A., SAINT-MARTORY (FR)

(21) Appl. No.: 10/557,748

(22) PCT Filed: May 10, 2004
(86) PCT No.: PCT/EP04/04963

§ 371 Date: Nov. 18, 2005

(30) Foreign Application Priority Data
May 16, 2003 (FR) 0305873

(51) Int. Cl. A61K 36/48; A61K 8/97

(52) U.S. Cl. 424/74; 424/757

(57) ABSTRACT

The invention provides cosmetic and dermatological preparation which contains an extract of plants of the genus Adenanthera and particularly seeds and hulled seed of the plants Adenanthera pavonina. The extracts are useful for inclusion in skin and hair treating preparations particularly for treating skin, scalp and hair for soothing irritations, preventative and treatment of skin aging, reducing inflammation and treatment of itching and flaking of the scalp.

### FIELD OF THE INVENTION

This invention relates generally to cosmetic preparations and, more particularly, to preparations containing special plant extracts and to the use of these plant extracts in cosmetic preparations, for example for the treatment of skin.

### PRIOR ART

Today, cosmetic preparations are available to the consumer in a variety of combinations. Consumers not only expect these preparations to have a certain care effect or to eliminate a certain deficiency, they are also increasingly demanding products which combine several properties and thus show an improved performance spectrum. There is a particular interest in substances which positively influence the technical properties of the cosmetic product, such as storage stability, light stability and formulatability and which, at the same time, represent active principles that impart, for example, caring, irritation-inhibiting, inflammation-inhibiting and/or UV-protecting properties to the skin and hair. In addition, consumers demand high dermatological compatibility and, above all, the use of natural products. The function of the skin as an organ enveloping the organism lies in sealing and mediating functions with respect to the environment. Various biochemical and biophysical systems serve to maintain the integrity of this exposed organ. For example, an immune system protects the skin against damage by pathogenic microorganisms, the melanin-forming system regulates pigmentation and protects the skin against radiation damage, a lipid system produces lipid micelles which control the transdermal water loss and a regulated keratin synthesis contributes to the mechanical resistance of the horny layer. The systems mentioned are based on complex chemical processes which are kept going inter alia by enzymes and controlled by enzyme inhibitors. Even the slightest inhibition or disinhibition of these biochemical systems is reflected in perceptible changes to the skin. However, the visible and noticeable condition of the skin is a measure of beauty, health and youthfulness; maintaining it is a general goal of skin care. The human skin generally reacts to exogenous, i.e. external, stress factors, such as UV radiation, ozone or other harmful substances present in the atmosphere (air pollutants), in the form of mild or relatively serious irritation. In particular, the skin is damaged by the oxygen radicals and non-specific proteinases released in irritation reactions. This can have an adverse effect, for example, on the appearance or the elasticity or the barrier functions of the skin. For example, endogenous proteases mobilized in the event of inflammatory processes and immune reactions, such as trypsin, elastases, collagenases and plasmin for example, can attack the skin and, above all, its structural proteins, such as collagen and elastin. The use of protease inhibitors from vegetable sources and, more particularly, serine protease inhibitors, such as trypsin inhibitors, was described, for example, in U.S. Pat. No. 4,906,457 for preventing cancer caused by UV radiation or for preventing flaking as an anti-desquamant in EP 0 975 324 or against changes in skin pigmentation in WO 99/04752. Elastase-inhibiting protein fractions from vegetable extracts and their use as inflammation-inhibiting, hydrating, elasticity-increasing, proteinase-inhibiting active principles are described in EP 532 465. Plasmin-inhibiting effects of plant extracts are disclosed in U.S. Pat. No. 4,066,507, JP-A 2002080359, JP-A 2001354582, JP-A 2001240551, JP-A 2001122728, JP-A2000327555, EP-A 0953341, WP 98/24474, JP-A 09020643, JP-A 09020642, JP-A 09020641, JP-A 09020640, EP-A 0567816, EP-A 0223254, JP-A200280359, JP-A9725214, JP-A 9612586, JP-A 8993509 and CS 124782. For many years now, plant extracts have been used in various cultures both for medicinal and for cosmetic purposes. Again and again, new plants are being extracted and the extracts tested for their cosmetic effects in order to find other plants with new or modified action spectrums. Many plants whose worth was not yet known and which were regarded as exotic and unimportant are now being widely used inter alia in cosmetic products

### DESCRIPTION OF THE INVENTION

The problem addressed by the present invention was to provide cosmetic and/or dermatological preparations which would meet the requirements cosmetic formulations are expected to satisfy, such as storage stability and dermatological compatibility, and which, besides care properties, would above all have improved protective properties for the human skin and/or scalp and/or hair, for example against UV radiation and other environmental influences, and, at the same time, a preventative and curative effect against signs of ageing of the skin and which could be used to inhibit inflammation.

Another problem addressed by the present invention was to provide preparations which would contain active principles from renewable raw materials and which, at the same time, could be widely used as a care component in skin- and hair-care preparations.

The present invention relates to cosmetic and/or dermatological preparations containing an extract of the seeds of plants of the genus Adenanthera.

In one particularly preferred embodiment, the extracts according to the invention are extracts of the seeds of the plant Adenanthera pavonina, more particularly an extract of hulled seeds synonymous with the embryos of the seeds.

The extracts are preferably used in quantities of 0.001 to 25% by weight, preferably in quantities of 0.05 to 5% by weight and more particularly in quantities of 0.1 to 0.5% by weight, expressed as dry weight based on the total quantity of the preparations, with the proviso that the quantities shown add up to 100% by weight with water and optionally other auxiliaries and additives.

The terms preparation, composition and care component are used synonymously in the present specification.

It has surprisingly been found that the extracts of seeds of plants of the genus Adenanthera and, more particularly, extracts of seeds of the plant Adenanthera pavonina satisfy the requirements stated above in excellent fashion. The extracts or the active principles present in them are readily obtainable and represent extremely efficient plasmin inhibitors. Accordingly, the substances are particularly suitable for protection against skin irritations, inflammations and the harmful effects of UV-A, UV-B and IR rays which lead to ageing and wrinkling of the skin.

Adenanthera Pavonina

The extracts to be used in accordance with the invention are obtained from seeds of plants of the genus Adenanthera and more especially from seeds of the plant Adenanthera pavonina. The genus Adenanthera includes eight species, above all in tropical Asia, in Australia and in the Pacific region. Besides Adenanthera pavonina, there is also the species Adenanthera abrosperma. Adenanthera pavonina is also known under the synonyms Adenanthera gersenii Scheffer or Adenanthera abrosperma, agati petite feuille, Circassian tree, coral wood, red bead tree, red wood, pearl tree, Indian coral tree or red sandalwood tree. Botanically, it belongs to the family of Leguminosae or Fabaceae. This plant is a 6 to 15 meters tall, quick-growing tree with grayish-brown bark. The leaves are found on spirally arranged, 20 to 30 cm long branches and are elliptical in shape and 5 to 10 cm long. The flowers are light yellow and fragrant on 5 to 15 cm long stems. The seeds are bright scarlet red and very uniform in diameter and in weight; four seeds correspond to 1 gram and each seed is 8 mm in diameter. The plant is native to Sri Lanka, Burma, Indochina, Surinam, South China, Thailand, Malaysia and Indonesia. It is cultivated as an ornamental plant and also as a shade plant for coffee or spice plants, as a fuelwood source or as wood for making furniture. The seeds are often used for ornamentation, but were also used in ancient India as a measure for gold. In Indian medicine, the crushed seeds, partly mixed with honey, were used for the treatment of suppurating and inflamed abscesses. The seed extract is used for the treatment of lung inflammations and chronic eye diseases.

A trypsin/chymotrypsin inhibitor was isolated from the seeds by extraction with 0.01 M hydrochloric acid, followed by chromatographic separation techniques [Natural Plant Enzyme Inhibitors. Isolation and Characterisation of a Trypsin/Chymotrypsin Inhibitor from Indian Red Wood (Adenanthera pavonina) Seeds; K. Sudhakar Prabhu and Thillaisthanam N. Pattabiraman; J. Sri. Food Agric. 1980, 31, No. 10, 967-980]. The size of the extracted inhibitor was determined by gel chromatography as 24,000 Da. A trypsin inhibitor was also isolated by Richardson et al. by extraction of the acetone-defatted seeds with 0.1 M sodium phosphate buffer (pH 7.6) in 1% NaCl [The amino acid sequence and reactive (inhibitory) site of the major trypsin isoinhibitor (DE5) isolated from seeds of the Brazilian Carolina tree (Adenanthera pavonina L.); M. Richardson, F. A. P. Campos, J. Xavier-Filho, M. L. R. Macedo, G. M. C. Maia and A. Yarwood; Biochimica and biophysica Acta, 1986, 872, No. 1-2, 134-146].

Eight isoenzymes were identified. They all had a size of ca. 21,000 Da and a large α-chain (Mr 16,000) and a relatively small β-chain (Mr 5,000) linked by a disulfide bridge. The amino acid sequence and the reactive center of the DE5 isoenzyme showed a strong accordance with the Kunitz-type protease inhibitors from soybeans or other leguminous seeds.

Like chymotrypsin, elastin and plasmin, trypsin is a serine protease.

Extraction

The extracts may be prepared by methods known per se, i.e. for example by aqueous, alcoholic or aqueous/alcoholic extraction of the seeds. Suitable extraction processes are any of the conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux. Percolation is advantageous for industrial use. The starting material normally consists of seeds which may be hulled and mechanically size-reduced before extraction. Any size reduction methods known to the expert, for example freeze grinding, may be used. After size-reduction of the seeds, the embryo may preferably be freed from the hull by sieving. Preferred solvents for the extraction process are organic solvents, water (preferably distilled water kept at room temperature) or mixtures of organic solvents and water, more particularly low molecular weight alcohols with more or less high water contents. The extracts according to the invention may be obtained from the leguminosae seeds mentioned by preferably grinding the hulled seeds, optionally extracting the powder obtained with an organic solvent or solvent mixture, drying and extracting the powder thus defatted with water or an aqueous electrolyte solution at a pH of 2 to 10 and preferably at a pH of 5 to 6, adjusting the extract to a pH of 5 to 7, preferably 5.2, concentrating in vacuo, clear-filtering the concentrate after addition of a filter aid, for example Celite, or centrifuging or freeze-drying the concentgrate. Extraction with distilled water at a pH of 5 to 6 is preferred. The proteins therefrom can be enriched and graded according to size by membrane enrichment in an ultrafiltration cell, for example from Amicon (10,000 Da cutoff or 15,000 Da cutoff).

The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of seeds are in the range from 10 to 30 and more particularly 13 to 25% by weight. The present invention includes the observation that the extraction conditions and the yields of the final extracts may be selected according to the desired application. These extracts, which generally have active substance contents (=solids contents) of 0.5 to 10% by weight), may be used as such, although the solvent may also be completely removed by drying, more particularly by spray or freeze drying. The extracts may also be used as starting materials for producing the pure active substances mentioned above unless they can be synthesized by a more simple and inexpensive method. Accordingly, the active substance content in the extracts may be from 5 to 100% by weight and is preferably from 50 to 95% by weight. The extracts themselves may be present as water-containing preparations and/or as preparations dissolved in organic solvents and as spray-dried or freeze-dried water-free solids. Suitable organic solvents in this connection are, for example, aliphatic alcohols containing 1 to 6 carbon atoms (for example ethanol), ketones (for example acetone), halogenated hydrocarbons (for example chloroform or methylene chloride), lower esters or polyols (for example glycerol or glycols).

Commercial Applications

The present invention also relates to the use of extracts of the seeds of plants of the genus Adenanthera, more particularly extracts of the seeds of the plant Adenanthera pavonina, for the production of cosmetic and/or dermatological preparations and more particularly for the production of treatment preparations for the skin, scalp and hair, in which they may be present in quantities of 0.001 to 25% by weight, preferably 0.05 to 5% by weight and, more particularly, 0.1 to 0.5% by weight, expressed as dry weight based on the total quantity of the preparations. The use of extracts of the hulled seeds is particularly preferred.

Other particular embodiments of the invention relate to the use of extracts of the seeds of plants of the genus Adenanthera, more particularly extracts of the seeds of the plant Adenanthera pavonina, for the production of cosmetic and/or dermatological preparations and more particularly for the production of treatment preparations for the skin, scalp and hair
with a soothing, relieving and irritation-inhibiting effect, more particularly against oxidative stress and/or air pollutants,
with a plasmin-inhibiting effect,
against ageing and wrinkling of the skin for the preventative or curative treatment of signs of skin ageing caused in particular by UV-A, UV-B and/or IR radiation,
for reducing inflammation of the skin, more particularly for the treatment of rosacea,
for the treatment of sensitive skin, more particularly for the treatment of dry skin,
against itching, more particularly against itching of the scalp
against scale formation, more particularly against dandruff on the scalp.

The extracts according to the invention have an irritation-inhibiting effect against oxidative stress for the skin, scalp or hair which can be triggered, above all, by UV or IR radiation, by high environmental pollution levels and by hormonal or biological effects on the skin, scalp or hair. The extracts according to the invention act against ageing of the skin and may be used for the preventative or curative treatment of signs of ageing of the skin. Another name for care preparations of this kind is anti-ageing preparations. These signs of skin ageing include, for example, any type of creasing or wrinkling. The treatments include slowing down of skin ageing processes. The ageing signs can have various causes. In particular, they are caused by UV and/or IR-induced damage to the skin. During inflammation or during the skin ageing process, proteases, such as elastase, collagenase and plasmin for example, are secreted from the skin by polymorphonuclear neutrophilic granulocytes or by macrophages. In another way, dermal fibroblasts in the elderly or, as a result of UV radiation, can secrete interstitial collagenase, so-called MMP-1 (matrix metallo proteinase) while UV-exposed keratinocytes produce a tissue plasminogen activator (t-PA) that splits plasminogen into plasmin. These proteases (elastase, collagenase and plasmin) catalyze the fragmentation of very important macromolecules of the skin, such as proteoglycan, collagen and elastin for example.

Plasmin is a human serine protease which plays a key role in wound healing. Plasmin degrades blood clots consisting of fibrin into soluble products, the fibrinopeptides, and promotes the migration of keratinocytes to cover an injury.

Plasminogen is the pro-enzyme which is activated by a protease to plasmin. This protease is urokinase which is secreted by activated keratinocytes during wound healing or during skin irritations or by inflammation of the skin. Plasminogen is released during inflammation by blood vessels with high permeability. The expression and secretion of urokinase is increased by UV-B radiation on the cells. In addition, plasminogen in extracellular matrix is transformed into plasmin that can then activate pro-MM3 which can then lead to the degradation of dermal glycoproteins, such as fibronectin, laminin and proteoglycan. Plasmin plays a key role in skin damage and hence in photoageing processes of the skin.

Accordingly, the plasmin-inhibiting effect of the extract according to the invention may be used to reduce inflammation of the skin or scalp and, more particularly, for the treatment of rosacea.

Rosacea is a hereditary, non-infectious skin disease in which the blood vessels widen and cause the skin to turn red. In certain phases, inflammation can also occur around the sebaceous glands. These inflammatory processes cause pimples and pustules. The skin disease rosacea—translated—means the same as “rosebud”. This alludes to the reddening of the face which is typical of rosacea. Besides this reddening, which is caused by widened blood vessels, changes to the nose can also occur through inflammation. Although the cause of rosacea has still not been fully elucidated, the basis is evidently the so-called rosacea diathesis. In other words, the tendency to react to certain stimuli by pronounced facial reddening which disappears again after a time. This reddening is also known as flush. The inflammation results in an increase in connective tissue which is visible as thickening of the skin. If these episodes remain untreated for long periods, so-called rhinophyma (“bulbous nose”) can develop. Inflammation of the eyelid rims and conjunctiva also frequently occurs in rosacea.

The extracts according to the invention are used for the production of skin and hair treatment preparations for the treatment of sensitive skin, more particularly dry skin, of which the typical feature is a low-fat, scaly, tender surface with small cracks and isolated inflamed regions.

The extracts according to the invention are used for the production of skin and hair treatment preparations for the treatment of itching, more particularly itching of the scalp. This itching can be caused by various factors such as, for example, insect bites, skin contaminants, hormonally or bacteriologically induced skin changes, air pollution and other environmental influences. On the scalp, the itching is often associated with dandruff. The extracts according to the invention are also used for the production of skin and hair treatment preparations against flaking and, more particularly, dandruff on the scalp. A suitable preparation for the treatment of flaking of the scalp is a hair shampoo or other hair care preparation, for example a hair rinse or hair spray.

Cosmetic, Pharmaceutical and/or Dermatological Preparations

The extracts according to the invention may be used for the production of cosmetic or dermatological preparations, such as for example hair shampoos, hair lotions, foam baths, shower baths, creams, gels, lotions, alcoholic and aqueous/alcoholic solutions, emulsions, wax/fat compounds, stick preparations, powders or ointments. These preparations may also contain mild surfactants, oil components, emulsifiers, pearlizing waxes, consistency factors, thickeners, superfatting agents, stabilizers, polymers, silicone compounds, fats, waxes, lecithins, phospholipids, UV protection factors, biogenic agents, antioxidants, deodorants, antiperspirants, antidandruff agents, film formers, swelling agents, insect repellents, self-tanning agents, tyrosine inhibitors (depigmenting agents), hydrotropes, solubilizers, perservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Emulsifiers

Suitable emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;
alkyl and/or alkenyl oligoglycosides containing 8 to 22 carbon atoms in the alk(en)yl group and ethoxylated analogs thereof;
addition products of 1 to 15 mol ethylene oxide onto castor oil and/or hydrogenated castor oil;
addition products of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
block copolymers, for example Polyethyleneglycol-30 Dipolyhydroxystearate;
polymer emulsifiers, for example Pemulen types (TR-1, TR-2) of Goodrich;
polyalkylene glycols and
glycerol carbonate.

Ethylene Oxide Addition Products
The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations.

Alkyl and/or Alkenyl Oligoglycosides
Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides
Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters
Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters
Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers
Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers
Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.
   Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silcas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat poly-peptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

UV Protection Factors

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxy-benzophenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol® 1789) or 1-phenyl-3-(4′-isopropylphenyl-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Biogenic Agents and Antioxidants

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Antioxidants interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, ⁻trihydroxy-butyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors
Basically, suitable germ inhibitors are any substances which act against gram-positive bacteria such as, for example, 4-hydroxybenzoic acid and salts and esters thereof, N-(4-chloro-phenyl)-N′-(3,4-dichlorophenyl)-urea, 2,4,4′-trichloro-2′-hydroxy-diphenylether(triclosan), 4-chloro-3,5-dimethylphenol, 2,2′-methylene-bis-(6-bromo-4-chlorophenol), 3-methyl-4-(1-methyl-ethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Odor Absorbers
Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of labdanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, lemon balm oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants
Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
non-aqueous solvents such as, for example, ethanol, propylene glycol and/or glycerol.
Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.
Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.
   Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are piroctone olamine (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, kojic acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and poly-ethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives

Suitable preservatives are, for example, phenoxyethanol, formal-dehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labdanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparations. The preparations may be produced by standard hot or cold processes and are preferably produced by the phase inversion temperature method.

### EXAMPLES

### Production Example H1

Adenanthera pavonina seeds were coarsely ground and the red hull was separated from the yellow embryos (cotyledons) by sieving. The embryos were finely ground and a fine powder, the embryo powder, was obtained.

The anti-trypsin activity of the embryo powder, as determined by Kakade et al.'s method, was 80.8 TUI/mg.

In a reactor, 30 g embryo powder were added to 300 ml distilled water. The mixture was homogenized using an Ultra-Thorax. The solution had a pH of 5.9. The solution was extracted for 1 hour at room temperature, the mixture was then centrifuged for 15 mins. at 5,000 G and the upper fatty phase was removed. The supernatant was passed through a 15 μm mesh filter. The pH of the solution thus obtained was adjusted with sulfuric acid to 5.0, resulting in the formation of a deposit. The suspension obtained was centrifuged for 15 mins. at 5,000 G. 230 ml of a yellow filtrate were obtained and then freeze-dried. 4.45 g lyophilizate were obtained, corresponding to a yield of 14.85%, based on the embryo powder. The lyophilizate had an anti-trypsin activity of 250 TUI/mg which corresponds to a 3.1-fold increase in activity by comparison with the embryo powder.

### Production Example H2

Batch A

22 g of the embryo powder obtained in accordance with Example 1 were added to 220 ml distilled water. The mixture was homogenized using an Ultra-Thorax. The solution had a pH of 5.9. The pH of the solution was adjusted to 5.2 with 4 n sulfuric acid. The solution was then extracted for 1 hour at room temperature at that pH value and the mixture was then centrifuged for 15 mins. at 5,000 G. After the addition of Celite, the supernatant was passed through a 45 μm mesh filter and then through a 0.2 μm mesh filter. 102.1 ml of a yellow filtrate were obtained and then freeze-dried. 3.06 g lyophilizates were obtained, corresponding to a yield of 13.91%, based on the embryo powder. The lyophilizate had an anti-trypsin activity of 314 TUI/mg which corresponds to a 3.9-fold increase in activity by comparison with the embryo powder.

Batch B

The extract was prepared in the same way as Batch A. An extract yield of 21.9% was obtained from 58 g embryo powder: anti-trypsin activity 251.8 TUI/mg which corresponds to a 3.1-fold increase in activity by comparison with the embryo powder.

Batch C

The extract was also prepared in the same way as Batch A except that the ratio of embryo powder to water was changed from 1:10 to 1:15 and extraction was carried out for 1.5 h. 16.13 g lyophilizate were obtained from 70 g embryo powder which corresponds to a yield of 23.04%, based on the embryo powder. The anti-trypsin activity of the lyophilizate amounted to 287.2 TUl/mg which corresponds to a 3.6-fold increase in activity by comparison with the embryo powder.

### Production Example 3

150 ml of an extract prepared in accordance with Example 2, batch B, were introduced into an ultrafiltration cell (Amicon Model 8200, 200 ml) equipped with a membrane having a 10,000 Da cutoff (Amicon ref. YM 10). The extract was concentrated by the membrane to a volume of 50 ml and another 50 ml distilled water were added. The solution was ultrafiltered and 50 ml filtrate were obtained. The permeate obtained was freeze-dried and 1 g lyophilizate were obtained. The lyophilizate had an anti-trypsin activity of ca. 450 TUI/mg which corresponds to a 1.8-fold increase in activity by comparison with the extract used and to a 5.6-fold increase by comparison with the embryo powder.

### Production Example 4

2,600 ml of an extract prepared in accordance with Example 2 were introduced by ultrafiltration (concentration and diafiltration) into a TIA ultrafiltration module equipped with 2 Carbosep membranes (Tech-Sep membrane with a 15,000 Da cutoff, 80 cm² membrane). The temperature was kept at 25° C. by a heat exchanger. After the first ultrafiltration, 1,300 ml filtrate were obtained to which 1,300 ml distilled water were added for the diafiltration step. The filtrate obtained was freeze-dried. The lyophilizate had an anti-trypsin activity of ca. 457 TUI/mg which corresponds to a 2-fold increase in activity by comparison with the extract used and to a 5.65-fold increase by comparison with the embryo powder.

Anti-Protease Activity Test

During inflammation or during the skin ageing process, proteases, such as elastase, collagenase and plasmin for example, are secreted from the skin by polymorphonuclear neutrophilic granulocytes or by macrophages. In another way, dermal fibroblasts in the elderly or, as a result of UV radiation, can secrete interstitial collagenase, so-called MMP-1 (matrix metallo proteinase) while UV-exposed keratinocytes produce a tissue plasminogen activator (t-PA) that splits plasminogen into plasmin. These proteases (elastase, collagenase and plasmin) catalyze the fragmentation of very important macromolecules of the skin, such as proteoglycan, collagen and elastin for example.

### EXAMPLE

### Inhibition of Elastase Activity

Elastase is a protease which is secreted either during inflammation by the leucocytes or as a result of UV-A damage by the fibroblasts and is jointly responsible for the degradation of dermal macromolecules, such as collagen and elastin for example, and hence for ageing of the skin. In order to test the effectiveness of the plant extract in inhibiting the release of elastase, pancreas elastase (a serine protease) was investigated and, as substrate, elastin was marked with a chromogenic synthetic substrate. The system was incubated with the active principles for 30 minutes at room temperature and then, after centrifuging, the optical density of the dye was determined at 410 nm. The extracts were used in a quantity of 0.3% by weight. The results are set out in Table 1 where they are expressed relative to a control as standard (=0%); a1-antitrypsin was used as the standard.

### EXAMPLE

### Inhibition of Plasmin Activity

Background: Plasmin is a human serine protease which plays a key role in wound healing. Plasmin degrades blood clots consisting of fibrin into soluble products, the fibrinopeptides, and promotes the migration of keratinocytes to cover an injury. Plasminogen is the pro-enzyme which is activated by a protease to plasmin. This protease is urokinase which is secreted by activated keratinocytes during wound healing or during skin irritations or by inflammation of the skin. Plasminogen is released during inflammation by blood vessels with an increased permeability. The expression and secretion of urokinase is increased by UV-B radiation on the cells. In addition, plasminogen in extracellular matrix is transformed into plasmin that can then activate pro-MM3 which can then lead to the degradation of dermal glycoproteins, such as fibronectin, laminin and proteoglycan. Plasmin plays a key role in skin damage and hence in photoageing processes of the skin.

Method: Human plasmin obtained from Sigma was mixed with the extract in a quantity of 0.3% by weight and incubated for a few minutes at 20° C. Natural casein marked with a quenched fluorescence probe (Interchim natural) was then added. The protease-catalyzed hydrolysis degraded the quenching and produced a fluorescence signal. The proportion of hydrolyzed substrate was determined by measuring the increased green fluorescence over a period of 30 minutes. The more active the plasmin, the more substrate is hydrolyzed and the higher the fluorescence intensity becomes. The inhibition of enzyme activity was evaluated by comparison with a control and a reference substance SBT1 (Sigma).

TABLE 1
Elastase and plasmin inhibition

| | Elastase | Plasmin |
|---|---|---|
| | inhibition [%] | inhibition [%] |
| Control | 0 | 0 |
| Extract of Example 1 | 29 | 66 |
| Extract of Example 2 (batch A) | 31 | 71 |
| Extract of Example 2 (batch B) | 19 | 72 |
| Extract of Example 2 (batch C) | 15 | 69 |
| Extract of Example 3 | 20 | 82 |
| Standard | α1-anti-trypsin | SBTI IC50 = |
| | IC50 = 0.13 mg/ml | 0.006% |

The results show that the various extracts of Adenanthera pavonina seeds are capable of inhibiting elastase and especially pancreas elastase and plasmin, but not to the same extent. The inhibition of plasmin is comparably higher than that of elastase.

The IC50% value of plasmin was determined on the basis of these results.

TABLE 2
IC50% inhibition values/control (mean of 2 tests)

| | | | | | |
|---|---|---|---|---|---|
| Concentration [% by wt.] | 0 | 0.03 | 0.1 | 0.3 | IC50% |
| Extract of Example No. | | | | | |
| 1 | 0 | 11 ± 2 | 39 ± 1 | 66 ± 1 | 0.182% |
| 2 (batch A) | 0 | 11 ± 2 | 47 ± 0 | 71 ± 2 | 0.125% |
| 2 (batch B) | 0 | 13 ± 0 | 44 ± 2 | 72 ± 2 | 0.143% |
| 2 (batch C) | 0 | 18 ± 1 | 42 ± 5 | 60 ± 5 | 0.159% |
| 3 | 0 | 34 ± 0 | 61 ± 0 | 82 ± 1 | 0.072 |
| 4 | 0 | 38 ± 8 | 62 ± 9 | — | 0.065 |

These results show that the increase in the anti-plasmin activity (a reduction in the IC50% value) is parallel to the enrichment of the trypsin inhibitor determined in the Production Examples.

Tables 3 to 6 contain a number of Formulation Examples.

TABLE 3
Examples of cosmetic preparations (water, preservative to 100% by weight)

| Composition (INCI) | 1 | 2 | 3 | 4 | 5 | 6 | 7 | 8 | 9 | 10 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin VL 75 | — | — | — | — | — | 3.5 | — | — | 2.5 | — |
| Polyglyceryl-2 Dipolyhydroxystearate (and) | | | | | | | | | | |
| Lauryl Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Bisabolol | 1.2 | 1.2 | 1.2 | 1.2 | 1.2 | 1.2 | 1.2 | 1.2 | 1.2 | 1.2 |
| Extract of Example 2 (batch A, B or C) | 0.05 | 0.05 | 0.05 | 0.05 | 0.05 | 0.05 | 0.05 | 0.05 | 0.05 | 0.05 |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

(1) w/o sun protection cream, (2-4) w/o sun protection lotion, (5, 8, 10) o/w sun protection lotion, (6, 7, 9) o/w sun protection cream

TABLE 3
Formulations for conditioners
Cosmetic preparations conditioners (water, preservative to 100% by weight)

| | 12% | 12% | 13% | 14% | 15% | 16% |
|---|---|---|---|---|---|---|
| | by | by | by | by | by | by |
| Composition (INCI) | wt. | wt. | wt. | wt. | wt. | wt. |
| Dehyquart ® A | 4.0 | 4.0 | | | 3.0 | |
| Cetrimonium Chloride | | | | | | |
| Dehyquart ® A | | | 1.2 | 1.2 | | 1.0 |
| Dococoylmethylethoxymonium Methosulfate (and) Propyleneglycol | | | | | | |
| Eumulgin ® B2 | 0.8 | | — | 0.8 | — | 1.0 |
| Ceteareth-20 | | | | | | |
| Eumulgin ® VL 75 | — | 2.0 | 2.0 | — | 0.8 | — |
| Lauryl Glucoside (and) Polyglyceryl-2 Polyhydroxystearate (and) Glycerin | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Coco-Glucoside (and) Gyceryl Oleate | | | | | | |
| Cetiol ® J 600 | — | 0.5 | — | 1.0 | — | 1.0 |
| Oleyl Erucate | | | | | | |
| Eutanol ® G | — | — | 1.0 | — | — | 1.0 |
| Octyldodecanol | | | | | | |
| Generol ® 122 N | — | — | — | — | 1.0 | 1.0 |
| Soya Sterol | | | | | | |
| Extract of Examples 1 to 4 | 1.0 | 1.0 | 1.0 | 1.0 | 1.0 | 1.0 |
| Copherol ® 1250 | — | — | 0.1 | 0.1 | — | — |
| Tocopheryl Acetate | | | | | | |

(11-14) Hair rinse, (15-16) hair treatment

TABLE 3
Cosmetic preparations shampoo (water, preservative to 100% by weight)

| Composition (INCI) | 17 | 18 | 19 | 20 | 21 | 22 |
|---|---|---|---|---|---|---|
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth Sulfosuccinate | | | | | | |
| Plantacare ® 818 | 4.0 | | | | | |
| Coco Glucosides | | | | | | |
| Plantacare ® 2000 | | 4.0 | | | | |
| Decyl Glucoside | | | | | | |
| Plantacare ® PS 10 | | | 20.0 | | | |
| Sodium Laureth Sulfate (and) Coco Glucosides | | | | | | |
| Dehyton ® PK 45 | 5.0 | | | 10.0 | | 10.0 |
| Cocamidopropyl Betaine | | | | | | |
| Gluadin ® WK | | | | | 8.0 | |
| Sodium Cocyl Hydrolyzed Wheat Protein | | | | | | |
| Lamesoft ® PO 65 | — | — | — | — | 2.0 | 2.0 |
| Coco-Glucoside (and) Glyceryl Oleate | | | | | | |
| Nutrilan ® Keratin W | 5.0 | — | — | — | | — |
| Hydrolyzed Keratin | | | | | | |
| Gluadin ® W 40 | — | 2.0 | — | 2.0 | — | — |
| Hydrolyzed Wheat Protein | | | | | | |
| Euperlan ® PK 3000 AM | — | — | — | 3.0 | 3.0 | — |
| Glycol Distearate (and) Laureth-4 (and) Cocamidopropyl Betaine | | | | | | |
| Panthenol | — | — | — | — | — | 0.2 |
| Extract of Examples 1 to 4 | 1.0 | 1.0 | 1.0 | 1.0 | 1.0 | 1.0 |
| Arlypon ® F | 1.5 | — | — | — | — | — |
| Laureth-2 | | | | | | |
| Sodium Chloride | — | 1.6 | 2.0 | 2.2 | — | 3.0 |

TABLE 4
Soft cream formulations K1 to K7
(All quantities in % by weight, based on the cosmetic preparation)

| INCI name | K1 | K2 | K3 | K4 | K5 | K6 | K7 | C1 |
|---|---|---|---|---|---|---|---|---|
| Glyceryl Stearate (and) Ceteareth-12/20 (and) | 8.0 | 8.0 | 8.0 | 8.0 | 8.0 | 8.0 | 8.0 | 8.0 |
| Cetearyl Alcohol (and) Cetyl Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 3.0 | 3.0 | 3.0 | 3.0 | 3.0 | 3.0 | 3.0 | 3.0 |
| Glycerin (86% by weight) | 3.0 | 3.0 | 3.0 | 3.0 | 3.0 | 3.0 | 3.0 | 3.0 |
| Extract of Examples 1 to 4 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 5
Night cream formulations K8 to K14
(All quantities in % by weight, based on the cosmetic preparation)

| INCI name | K8 | K9 | K10 | K11 | K12 | K13 | K14 | C2 |
|---|---|---|---|---|---|---|---|---|
| Polyglyceryl-2 Dipolyhydroxystearate | 4.0 | 4.0 | 4.0 | 4.0 | 4.0 | 4.0 | 4.0 | 5.0 |
| Polyglyceryl-3 Diisostearate | 2.0 | 2.0 | 2.0 | 2.0 | 2.0 | 2.0 | 2.0 | 2.0 |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl Isononanoate | 8.0 | 8.0 | 8.0 | 8.0 | 8.0 | 8.0 | 8.0 | 8.0 |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% by weight) | 5.0 | 5.0 | 5.0 | 5.0 | 5.0 | 5.0 | 5.0 | 5.0 |
| Extract of Examples 1 to 4 | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

TABLE 6
W/O body lotion formulations K15 to K21.
(All quantities in % by weight, based on the cosmetic preparation)

| INCI name | K15 | K16 | K17 | K18 | K19 | K20 | K21 | C3 |
|---|---|---|---|---|---|---|---|---|
| PEG-7 Hydrogenated Castor Oil | 7.0 | 7.0 | 7.0 | 7.0 | 7.0 | 7.0 | 7.0 | 7.0 |
| Decyl Oleate | 7.0 | 7.0 | 7.0 | 7.0 | 7.0 | 7.0 | 7.0 | 7.0 |
| Cetearyl Isononanoate | 7.0 | 7.0 | 7.0 | 7.0 | 7.0 | 7.0 | 7.0 | 7.0 |
| Glycerin (86% by weight) | 5.0 | 5.0 | 5.0 | 5.0 | 5.0 | 5.0 | 5.0 | 5.0 |
| MgSO₄•7H₂O | 1.0 | 1.0 | 1.0 | 1.0 | 1.0 | 1.0 | 1.0 | 1.0 |
| Extract of Examples 1 to 4 | 1.5 | 1.5 | 1.5 | 1.5 | 1.5 | 1.5 | 1.5 | — |
| Tocopherol | | 0.5 | | | | | | |
| Allantoin | | | 0.2 | | | | | |
| Bisabolol | | | | 0.5 | | | | |
| Chitosan (Hydagen CMF) | | | | | 10.0 | | | |
| Deoxyribonucleic acid¹⁾ | | | | | | 0.5 | | |
| Panthenol | | | | | | | 0.5 | |

| | |
|---|---|
| Water | to 100 |

¹⁾Deoxyribonucleic acid: molecular weight ca. 70,000, purity (determined by spectrophotometric measurement of absorption at 260 nm and 280 nm): at least 1.7

All substances with the registered trade mark symbol ® used and listed in Tables 3 to 6 are marks and products of the COGNIS Group.

1-10. (canceled)

11. A cosmetic and dermatological preparation containing an extract of the seeds of plants of the genus Adenanthera.

12. The preparation as claimed in claim 11 containing an extract of seeds of the plant Adenanthera pavonina.

13. The preparation as claimed in claim 11 containing an extract of hulled seeds of the plant of the genus Adenanthera.

14. The preparation as claimed in claim 12 containing an extract of the hulled seeds of the plant Adenanthera pavonina.

15. The preparation as claimed in claim 11 containing 0.001% to 5% by weight of the extract, expressed as dry weight based on the total quantity of the preparation, with the proviso that the quantities shown add up to 100% by weight with water and optionally other auxiliaries and additives.

16. A preparation as claimed in claim 12 containing 0.001% to 5% by weight of an extract of hulled seeds of the plant Adenanthera pavonina, expressed as dry weight based on the total quantity of the preparation, with the proviso that the quantities shown add up to 100% by weight with water and optionally other auxiliaries and additives.

17. A method of treating at least one member selected from the group consisting of the skin, scalp and hair which comprises contacting at least one member with a composition comprising an extract of a plant of the genus Adenanthera.

18. The method of claim 17 wherein the extract comprises an extract of the hulled seeds of the plant Adenanthera pavonina.

19. A method of soothing and inhibiting irritation of skin and hair due to oxidative stress and air pollutants which comprises contacting the skin or hair with a composition of claim 11.

20. The method of claim 19 wherein the extract comprises an extract of hulled seeds of the plant Adenanthera pavonina.

21. A method for treatment against aging and wrinkling of the skin which comprises contacting the skin with a composition of claim 11.

22. The method of claim 21 wherein the extract comprises an extract of hulled seeds of the plant Adenanthera pavonina.

23. A method for reducing inflammation of the skin and treatment of rosacea which comprises contacting the affected area of the skin with the composition of claim 11.

24. The method of claim 23 wherein the extract comprises an extract of hulled seeds of the plant Adenanthera pavonina.

25. A method for treating itching of the scalp and dandruff which comprises applying to the scalp the composition of claim 11.

26. The method of claim 25 wherein the extract comprises an extract of hulled seeds of the plant Adenanthera pavonina.

27. A method for inhibiting plasmin in the scalp and skin by applying to the scalp and skin a plasmin inhibiting amount of the composition of claim 11.

28. The method of claim 27 wherein the extract comprises an extract of hulled seeds of the plant Adenanthera pavonina.
